(19) United States
(12) Patent Application Publication
(10) Pub. No.: US 2004/0097715 A1
(43) Pub. Date: May 20, 2004

(54) Electropulsing process for producing proteins

(76) Inventors: Justin Teissie, Ramonville (FR); Valentina Ganeva, Sophia (BG); Bojidar Galutzov, Sophia (BG)

Correspondence Address: YOUNG & THOMPSON, 745 SOUTH 23RD STREET 2ND FLOOR, ARLINGTON, VA, 22202

(21) Appl. No.: 10/419,188

(22) Filed: Apr. 21, 2003

Related U.S. Application Data
(63) Continuation of application No. PCT/FR01/03261, filed on Oct. 19, 2001.

(30) Foreign Application Priority Data
Oct. 19, 2000 (FR) 00/13415

(51) Int. Cl. C07K 1/04; C12P 21/06; C12N 1/18; C12N 1/21

(52) U.S. Cl. 530/412; 435/69.1; 435/252.3; 435/254.2

(57) ABSTRACT

The present invention concerns a process for producing proteins in which a stream of liquid medium comprising at least one yeast, bacterium or mammalian cell undergoes electropulsing, and the proteins that are liberated are recovered. The process comprises an electropulsing step followed by an incubation step.

The present invention relates to a process for producing proteins of interest.

Micro-organisms, namely bacteria, fungi, plant cells, insect ca-ells and animal cells, and in particular yeasts, can produce proteins, but it can prove difficult to recover certain products from those micro-organisms, in particular macromolecules such as proteins.

It is known that proteins of interest, namely natural proteins, can be extracted from yeast using chemical and mechanical methods.

When using the usual mechanical methods constituted by grinding and lysis under pressure, it is impossible not to destroy the yeast vacuoles; liberation of the proteases contained in the vacuoles causes problems with purity and thus with purification of the finished product.

Further, chemical methods also result in the destruction of vacuoles and employ detergents or proteolysis agents, which are added to the chemical products used and which contaminate the medium. This also results in problems with subsequent purification.

Further, some of those chemical agents deactivate the enzymes.

The present invention concerns a novel approach in which an electropulsing treatment is applied to a suspension of flowing cells followed by incubation in a suitable medium. The cells can be yeasts, but can also be bacteria to which the electropulsing can be applied to a suspension in pure water, for example, followed by relatively long incubation in a saline medium, without the need for carrying out a pre-treatment of the bacterial culture with the aim of modifying it mechanically, chemically or biologically. It can also be adapted to mammalian cells as will become apparent in the detailed description.

The present invention concerns a process for producing proteins, in which a stream of liquid medium comprising at least one yeast, bacterium or mammalian cell undergoes electropulsing, and the proteins that are liberated are recovered. As will become clear, the term “electropulsing” means a process in which the cells are subjected to a series of electrical impulses.

The present invention overcomes the problems mentioned above. In accordance with the invention, the electrical treatment does not cause cells to disintegrate and thus, cell fragments do not appear in the medium after treatment. The entire contents of the cell are not released: the organites and the majority of the major cytoplasmic proteins are retained by the cell wall. The vacuoles are not affected and the proteases are not released. Purification of the proteins of interest is simplified and the process of the invention allows greater selectivity as regards the liberated products.

Studies concerning fixed bed electropulsing have been carried out, but the conditions described by V Ganeva, B Galutzov, FEMS, Microbiology Letters 174 (1999) 279-284 do not lead to the present process with the anticipated results.

The fixed bed process does not produce irreversible permeabilization of all of the treated cells. The Applicant has established that certain implementation conditions in the fixed bed process could lead to certain drawbacks with a flow system.

They mainly concern the conditions regarding the conductivity of the electropulsing medium. Finally, applied electropulsing treatments cause different effects with yeasts depending on whether they are carried out in a fixed bed or in a flow system, and in particular they cause different effects on cell permeabilization.

Enzyme deactivation can be avoided in a flow process, which means that substantial heating of the cell suspension can be avoided. This is highly advantageous, particularly as regards yield, as a subsequent cooling step and the use of the equipment necessary for this step are avoided.

In accordance with the invention, treating a highly concentrated suspension of cells can obtain optimum liberation without causing the medium to heat up.

Finally, it is possible to apply the process of the invention to large volumes and obtain large quantities of proteins of interest and a degree of purity that has been impossible to obtain until now by other methods, and in particular without adding protease inhibitors, using media with a simple composition.

In the process of the invention, applying a high density external electrical field to the cells, and in particular to cells in suspension, causes the appearance of a potential which adds to the transmembrane potential. When a critical level is reached, the cellular membrane becomes permeable. When the cells are moving (flow), they change position with respect to the electrodes. The pulses will have different regions of the surface as a target. This results in a larger permeabilized surface and a local level of structural change that differs from that of static cells (batch). For the defined parameters of the applied electrical field, such as the intensity, duration or number of pulses, the changes in the membrane caused by the pulses can become irreversible and result in liberation of the intracellular contents.

With yeasts having a wall, this outflow is controlled by the porosity of the wall and permeability to the different molecules, which depends on their dimensions.

The inventors have observed that the membranes of bacteria permeabilize for electric fields that are similar to the values employed for yeasts.

Permeabilization is a very rapid process which develops during the few microseconds during which the electrical impulses are applied, and which lasts for a period of a few minutes to hours after the electrical treatment.

It has been shown that the treatment applied to yeasts can cause liberation of the enzyme invertase, located in the periplasmic space. It appears that the porosity of the cell wall increases over and above the influence of the field on the plasma membrane.

The process of the invention allows the outflow and thus liberation and subsequent recovery of proteins secreted by the cell and which accumulate in the periplasmic space.

In other aspects, the invention also concerns proteins obtained by the process described herein and compositions comprising them.

Regarding the cells which can be treated by the process of the invention, all of the species that can produce heterologous or homologous proteins can be treated. The examples below are not limiting in scope.

Particular yeasts that can be mentioned are Saccharomyces, Kluyveromices, Pichia, Candida and Hansenula.

Bacteria that can be cited include Gram-negative bacteria, in particular E. coli.

Regarding mammalian cells, fibroblasts can be mentioned, in particular Chinese hamster cells.

In accordance with the invention, the cells are cultured then washed to eliminate ions from the medium, and then they undergo the electrical treatment and are then incubated in an incubation medium. Said cells are then separated from the supernatant. The proteins are purified by the usual means.

In accordance with the present invention, the electropulsing step is preceded by a culture step. Growth can be maintained until the exponential phase or until the stationary phase. Preferably, culture is up to the exponential phase, but positive results were also obtained in the stationary phase. The culture medium can therefore be selected to allow maximum expression of the desired protein, whether it is homologous or heterologous. The Applicant has demonstrated that the electroextraction efficiency is not influenced by the composition of the culture medium.

The cells in the culture medium are transferred to the pulsing medium after centrifuging the suspensions.

Preferably, the cells are in suspension in the liquid medium that undergoes electropulsing. The concentration of yeasts or bacteria in suspension in the liquid electropulsing medium is in the range approximately 10⁶ to 5×10⁹ cells/ml, preferably in the range about 5×10⁸ cells/ml to about 2×10⁹ cells/ml. The concentration of mammalian cells is preferably about 50 times lower.

Regarding the conditions for the electropulsing treatment, this can be carried out with field intensities of at least 1 kV/cm, in particular 1 to 10 kV/cm, preferably in the range 1 to 5 kV/cm, and in particular 2 to 4 kV/cm.

The pulse duration is preferably selected to be more than 0.01 ms (milliseconds), in particular more than 1 ms. It can be as long as 100 ms. Preferably, it is in the range 0.5 to 15 ms, in particular in the range 1 to 15 ms.

When high density electric fields are applied, a single pulse is generally applied. It is also possible to apply a series of consecutive pulses, said series having a duration of a few microseconds to a few milliseconds. The treatment applied to each cell lasts about 0.01 to 100 s, preferably 0.15 to 15 s.

The number of pulses received by each yeast, bacterium or mammalian cell is of the order of 1 to 100, preferably 5 to 20, more preferably 12 to 20, for example of the order of 10.

As an example, for 2 Hz, the pulse duration would be 3 ms and the field intensity would be 3 kV/cm; for 6 Hz, the duration would be 2 ms and the intensity would be 3.2 kV/cm, or 1 ms at 4.3 kV/cm.

As will be shown in the examples, series of pulses can be applied, for example a series of about fifteen pulses, one pulse having a duration of 0.1 to 4 ms, in particular 2 to 4 ms.

The pulse frequency is preferably more than 0.1 Hz, preferably 1 Hz. It can be as high as 100 Hz, or even 500 Hz. More preferably, it is in the range 1 to 100 Hz, in particular 1 to 10 Hz. Further, the product of the duration of each pulse by the frequency must be less than 1.

Regarding mammalian cells, the intensity of the applied electric field must be sufficient to allow the release of cytoplasmic proteins. In the case of the electrical parameters illustrated in the example, electric field intensities of more than 1 kV/cm must be used to allow liberation of cytoplasmic proteins into the external medium. It has been observed that the cell viability is 50% under the better salting-out conditions (1.25 kV/cm).

Regarding the pulse profile of the electric field, it is possible to use square, trapezoidal, triangular, sinusoidal, single and half-wave rectified sinusoidal waves, or with an exponential decay, unipolar, bipolar, symmetrical or asymmetrical; preferably, square waves are used.

In accordance with the invention, the conductivity of the electropulsing medium is low, preferably less than 2 mS/cm. It is selected to limit the Joule effect associated with electric pulses. Thus, it can be a simple saline medium. The electropulsing medium can also be deionized water for yeasts and bacteria.

After the electric field treatment, the liquid medium comprising the treated cells is incubated to encourage outflow of the proteins of interest.

The incubation medium can be the same as the pulsing medium, but it can also be modified. Transition from the pulsing medium to the incubation medium can be made by transferring the pulsed cells into a buffer containing an osmotic stabilizer.

The incubation medium as used in the invention can be a simple saline medium comprising at least one salt, for example a potassium phosphate. It is possible to use an incubation medium comprising at least one buffer such as potassium phosphate in an amount of 10 mM to 0.2 M, preferably 50-150 mM, an osmotic stabilizer such as glycerol in an amount of 0.05 to 1 M, preferably 0.2 to 0.5 M, and it can also comprise a reducing agent such as dithiothreitol in an amount of 0.01 to 0.2 mM—as an agent for preserving the stability of the extracted proteins—or it is also possible to use other products containing thiol groups (sulphohydric) such as mercaptoethanol or mercaptoethylamine.

Glycerol is frequently used as an osmotic protector, but in concentrations of 10% to 20%. In the present process, its presence is aimed at keeping the vacuoles intact, and its concentration is much lower (for example 2.8%), which facilitates subsequent purification of the liberated proteins.

When incubation is complete, the proteins can be purified by any known means for separating proteins from a liquid medium, for example by fixed volume centrifuging, or flow centrifuging, filtering, decanting or precipitation, preferably centrifuging or filtering.

Regarding the choice of culture media, incubation media and extraction media, in particular when treating yeasts, bacteria and mammalian cells, the skilled person could adapt the composition of said media as required.

Regarding yeasts and bacteria, the medium that undergoes electropulsing can be pure water and the culture medium can be a saline medium. Regarding mammalian cells, the culture medium must preferably be equilibrated osmotically, and the culture medium can be similar to the electropulsing medium. It is possible to use a medium with a low ion content; saccharose, for example, protects against osmotic shock.

The process of the invention can be applied to yeasts extracted from cytoplasmic enzymes such as DHA (dehydrogenase alcohol), PGK (phosphoglycerate kinase), HK (hexokinase), GAPDH (glyceraldehyde phosphate dehydrogenase), GLR (glutathione reductase), SOD (superoxide dismutase), and beta-galactosidase in the case of Kluiveromyces lactis.

In accordance with the invention, with yeasts the different stages of culture, incubation and extraction can have durations of the order of:

culture to the exponential phase: about 15 h;

culture to the stationary phase: 24 to 48 h;

separation of the cells from the culture medium, washing and dilution: 1 h;

electrical treatment of each volume traversing the pulsing chamber: a few seconds;

incubation aimed at liberating proteins after electropulsing to obtain a maximum: between 3 and 8 h;

separation of proteins: 10 min centrifuging at 2000 g.

In accordance with the invention, with bacteria the different stages of culture, incubation and extraction can have durations of the order of:

preculture overnight;

culture to the exponential phase: 4 to 10 hours;

separation of the cells from the culture medium, washing and dilution: 1 h;

electrical treatment of each volume traversing the pulsing chamber: a few seconds;

incubation aimed at liberating proteins after electropulsing to obtain a maximum: between 2 and 8 h;

separation of proteins: 10 min centrifuging at 2000 g.

In accordance with the invention, with mammalian cells, the different stages of culture, incubation and extraction can have durations of the order of:

culture to the exponential phase: a few days to obtain sufficient biomass;

separation of the cells from the culture medium, washing and dilution: 1 h;

electrical treatment of each volume traversing the pulsing chamber: a few seconds;

incubation aimed at liberating proteins after electropulsing to obtain a maximum: about 30 minutes;

separation of proteins: 10 min centrifuging at 100 g.

From the point of view of the equipment used to carry out the process, an electropulsing chamber connects a reservoir from which, following cultivation, washing and dilution, cells are sucked by the chamber, the flow causing them to pass between the electrodes connected to an electropulser. The chamber and electropulsing equipment can be modified: the electrical pulses can be applied at a frequency which is a function of the rate of flow, so that the cells receive a pre-defined number of pulses that are suitable as regards intensity and duration, for example 2 to 5 kV/cm and with a duration of 0.1 to 15 ms, for example 1 to 15 ms.

For similar durations, lower intensities of 0.5 to 5 kV/cm, preferably 1 to 4 kV/cm, can be used for mammalian cells.

At the outlet from the flow chamber, the stream of cells is diluted in the incubation medium and transmembrane release of proteins can take place. The proteins are then separated from the yeast, bacteria or mammalian cells by centrifuging or filtering. They are then purified.

The release of proteins from the micro-organism can be detected by the Coomassie blue test, which results in an umbrella determination of the proteins present in the extract, and is a reference method for determining the soluble proteins in a soluble extract. In the examples, the membrane proteins sedimented out during centrifuging. They were no longer present in the extract.

The enzymes can subsequently be concentrated and purified using known methods. Separation of the enzymes liberated from the cells of the supernatant is a routine step prior to subsequent purification. With electropulsing, since no cell fragmentation takes place, centrifuging at 1500-2000 g is sufficient for yeasts and bacteria, and 100 g suffices for mammalian cells. On an industrial scale, said separation can be carried out by continuous centrifuging or by filtering.

The electropulsing process was carried out on continuous streams of cell culture under the conditions given below.

### EXAMPLES OF PRODUCING PROTEINS BY ELECTROPULSING YEAST

Example a): Saccharomyces cerevisiae (strain FY-86) was cultivated in YPD medium (10 g/l of yeast extract, 20 g/l of peptone, 20 g/l of glucose) at 30° C. at 240 rpm. Growth was maintained to the exponential phase and to the stationary phase.

After culturing the yeast, the cells were separated from the culture medium by centrifuging at 2000 g for 5 minutes, washed twice with deionized water and diluted with deionized water to a concentration of 10⁹ cells/ml.

The pulse parameters were selected as follows: a series of fifteen pulses, each with a 3 ms duration, at a frequency of 4 Hz, the flow rate being 3 ml/min.

Just after treatment, the suspension was diluted 5 fold in the extraction medium, which is also the incubation medium in the present case. It comprised a final concentration of potassium phosphate buffer (0.1 M), glycerol (0.3 M) and 1 mM DTT.

The residence time in the extraction medium was of the order of 6 hours. The following electropulsing and incubation process aimed at extraction were carried out at ambient temperature.

The activity of the recovered proteins was determined using a specific test. The total amount of proteins liberated was determined using the Biorad test.

For Saccharomyces cerevisiae, it was seen that liberation of 80% of cytoplasmic proteins such as glyceraldehyde phosphate dehydrogenase, hexokinase, phosphoglycerate kinase, in a medium comprising potassium phosphate (0.084 M), glycerol (0.24 M) and 0.8 mM DTT, TOOK 4 hours.

Example b): a similar test was carried out under the same conditions as those given above, growth being maintained TO the stationary phase. Similar results to those obtained in a) were obtained.

Example c): Kluyvermyces lactis (strain 2209) was cultivated in YPL medium (10 g/l of yeast extract, 20 g/l of peptone, 20 g/l of lactose) and under the same conditions.

A very similar protocol to that described in a) was followed.

After culturing the yeast, the cells were separated by centrifuging at 2000 g for 5 minutes, washed twice with deionized water and diluted in deionized water to a concentration of 10⁹ cells/ml.

The pulse parameters were as follows: a series of fifteen pulses, each lasting 3 ms, at a frequency of 4 Hz, thus giving a flow rate that could reach 3 ml/min. The flow rate could be increased simply by modifying the geometric characteristics of the pulse chamber and the performances of the electropulser.

Just after treatment, the suspension was diluted 5-fold in the extraction medium, which again was the incubation medium, and comprised an initial buffer concentration of potassium phosphate (0.1 M), glycerol (0.3 M) and 1 mM DTT. The residence time in the extraction medium was of the order of 8 hours. The following electropulsing and incubation process for extraction was carried out at ambient temperature.

For Kluyveromyces lactis, liberation of cytoplasmic beta-galactosidase into a medium (phosphate (0.084 M), glycerol (0.24 M) and 1.6 mM DTT) took 7 to 8 hours.

After incubation, the cells were separated from the liquid containing the liberated proteins by centrifuging.

Example d): under the same conditions, Example a) was reproduced with the following pulse parameters: a series of 15 pulses, each lasting 3 ms, at a frequency of 50 Hz, the flow rate being 60 ml/min.

Example e): Example c) was repeated, with the exception that the pulse parameters were as follows: a series of 15 pulses, each lasting 3 ms, at a frequency of 6 Hz, and at a flow rate of up to 7.2 ml/min.

These data were compared with the results of conventional yeast lysis: for a), and for b), c), d) and e), there was no proteolytic degradation, while this occurred with other processes. Less than 5% to 10% of the enzymes were deactivated. This result demonstrates the superiority of the electrical method compared with that currently used on the laboratory scale or in an industrial process.

The profiles after polyacrylamine gel electrophoresis (PAGE), for which clear bands with no smearing were obtained, confirmed the absence of proteolytic degradation during the extraction process.

The following quantitative results were obtained for a), and for b), c), d) and e):

the recovered protein balance represented 45% to 50% of all of the cellular proteins (which could be obtained by enzymatic cell lysis or mechanical disintegration);

the balance of the enzymes liberated and analyzed is: phosphoglycerate kinase, glyceraldehyde phosphate dehydrogenase and hexokinase of the order of 80% to 90% of their cell content.

The specific activity of the enzymes in the supernatant from pulsed cells was 1.7 to 2 times higher than that obtained by mechanical grinding or enzymatic cell lysis. However, at the incubation stage, it was noticed that the recovered functional enzymes had been purified.

### EXAMPLES OF PRODUCING PROTEINS BY ELECTROPULSING BACTERIA

Culture:

50 ml of an overnight culture of E Coli (BL 21) was re-suspended in 450 ml of LB medium. The culture was incubated for 2 hours at 37° C., with agitation.

The optical density of the culture was E₆₆₀=0.7. It was centrifuged at 4000 rpm for 10 minutes. The residue was re-suspended in 500 ml of milliQ water (Millipore). Incubation was carried out at ambient temperature (25° C.) for 30 min.

It was centrifuged at 4000 rpm for 10 min and the residue was re-suspended in 45 ml of milliQ water. The two successive washes eliminated the major portion of the ions from the medium in which the bacteria were to be found.

Treatment Conditions:

The pulse chamber had a volume of 0.3 ml with a distance of 3 mm between the electrodes (parallel plates, stainless steel).

The flow rate was fixed at 2.4 ml/min using a peristaltic pump.

Series of pulses with an individual duration of 2 ms and a frequency of 3 Hz were applied continuously. This corresponded to an application of 22 pulses to each bacterium.

The tension in the generator was fixed at 1500 V, giving a field intensity of 5 kV/cm.

After the electrical treatment, 100 microlitres of the electrotreated bacterial suspension was re-suspended in 400 microlitres of 0.105 M PBS buffer, pH=7. The sample was then incubated at 37° C.

The control cells were treated in the same manner but the tension of the generator was fixed at 0 V.

After incubating for 5 hours, the cells were centrifuged in an Eppendorf centrifuge for 3 minutes (13000 g). The amount of proteins in the supernatant was determined.

Protein Determination:

An umbrella balance of the proteins present in the supernatant was then determined using a calorimetric test.

The sample was assayed by measuring the optical density at 595 nm of a mixture of 100 microlitres of supernatant, 700 microlitres of milliQ water and 200 microlitres of Bradford reagent (Biorad, USA).

The spectrophotometer reference was a mixture of 800 microlitres of milliQ water and 200 microlitres of Bradford reagent.

A reference range of proteins was produced using bovine albumin (Sigma, USA).

In conclusion, the test sample supernatant optical density was determined to be 0.570, giving 11.4 μg of protein, while that for the control was 0.103, giving 2.4 μg of protein. Under these measurement conditions, 9 μg of proteins were extracted from the bacterial sample evaluated by the electrical treatment followed by incubation for 5 h.

The quantity of proteins (9 μg) corresponded to the volume of the sample, i.e., 0.1 ml of supernatant, namely 25 μl of electrotreated bacterial suspension.

No pre-treatment of the bacterial culture aimed at mechanically, chemically or biologically modifying it was carried out.

### EXAMPLE OF PROTEIN PRODUCTION BY ELECTROPULSING MAMMALIAN CELLS

Culture:

Chinese hamster (CHO, WTT clone) cells are partially transformed cells. They were cultivated at 37° C. in suspension in glass spinner type flasks kept under gentle agitation (100 rpm) to avoid adhesion of the cells to the support. The culture medium used was minimum Eagle medium: MEM 0111 (Eurobio) complemented with 8% of foetal calf serum (Seromed), antibiotics (100 units per ml penicillin, 100 μg per ml streptomycin) and L-glutamine (0.58 mg per ml).

Electropulsing:

During cell electropulsing, the culture medium was replaced by pulse medium. For CHO cells, this was constituted by phosphate buffer (10 mM, pH 7.2), saccharose (250 mM) and MgCl₂ (1 mM) (conductance 1400 μS/cm±200 μS/cm; osmolality≈0.317 osmol/kg). This iso-osmotic medium with a low ionic strength limited the Joule effect associated with electric pulses due to its low conductance.

The cells were centrifuged at 100 g for 5 minutes. The supernatant was eliminated then the residue was taken up in the pulsing medium.

The cells were electropulsed at different fields with 10 pulses of 1 ms, delivered at a frequency of 1 Hz. The flow rate was 1.2 m./min. In the chamber, the electrodes were constituted by 2 parallel plates separated by an inter-electrode distance of 0.4 cm. The volume of the pulse chamber was 0.2 ml and the direction of the field was perpendicular to the mean flow direction.

After electropulsing, the cells were brought to ambient temperature to allow the cell membranes to return to their native state. 1.5 ml of cell suspension (1×10⁶ cells/ml) was recovered for each test. The cells were incubated for 10 minutes at ambient temperature then placed at 4° C. The cells were then centrifuged for 5 minutes at 100 g (800 rpm, C500 centrifuge, Jouan). The supernatant (≈1 ml) was removed and stored at 4° C.

Assay:

The proteins were assayed in the pulse buffer using a Biorad kit. A calibration scale was produced in parallel using bovine serum albumin (BSA, 1 mg/ml). A dilute solution of BSA (0.1 mg/ml) was used to prepare different dilutions for the calibration scale. 800 μl of solutions of the calibration scale and the different assays were supplemented with 200 μl of Biorad reagent. The optical density was read using a spectrophotometer at 595 nm. An umbrella balance of the electroextracted proteins was obtained.

Balance:

The results regarding the liberation of intracellular proteins as a function of the electric field intensity E (kV/cm) showed that for a field intensity of 1.25 kV/cm, the concentration of liberated proteins was 25 μg/ml and 50% of the cells remained viable. The viability was evaluated 24 h after the electrical treatment using the crystal violet colouring technique. Seeding culture dishes to quantify viability was carried out with the same number of cells (≈0.75×10⁶ cells/ml). 100% viability was expressed with respect to the OD obtained at 595 nm for cells that had not undergone electrical treatment but had been treated in a continuous flow.

1. A process for producing proteins in which a stream of liquid medium comprising at least one yeast, bacterium or mammalian cell undergoes electropulsing, and the proteins that are liberated are recovered.

2. A process according to claim 1, characterized in that the electropulsing step is followed by an incubation step.

3. A process according to claim 1 or claim 2, characterized in that the bacterial yeast(s) or mammalian cell(s) are in suspension in the liquid medium that undergoes electropulsing.

4. A process according to one of claims 1 to 3, characterized in that the concentration of yeast/s or bacterium/a in suspension in the liquid medium is in the range 10⁶ to 5×10⁹ cells/ml, preferably in the range 5×10⁸ to 5×10⁹ cells/ml, and the concentration of mammalian cells is in the range 2×10⁴ to 10⁸ cells/ml, preferably in the range 10⁷ to 10⁸ cells/ml.

5. A process according to one of claims 1 to 4, characterized in that the yeast is selected from species that can produce heterologous and homologous proteins, in particular Saccharomyces, Kluyveromyces, Pichia, Candida and Hansenula.

6. A process according to one of claims 1 to 5, characterized in that the liberated proteins are separated from the yeasts, bacteria or mammalian cells by filtering or centrifuging.

7. A process according to one of claims 1 to 6, characterized in that electropulsing is carried out with field intensities of at least 1 kV/cm.

8. A process according to one of claims 1 to 7, characterized in that the field intensity is in the range 1 to 10 kV/cm, preferably in the range 1 to 5 kV/cm.

9. A process according to one of claims 1 to 8, characterized in that the pulse duration is more than about 0.01 ms.

10. A process according to one of claims 1 to 9, characterized in that the pulse duration is less than 100 ms.

11. A process according to one of claims 1 to 10, characterized in that the pulse duration is in the range 0.5 to 15 ms.

12. A process according to one of claims 1 to 11, characterized in that the electropulsing treatment duration is about 0.01 to 100 s, preferably 0.15 to 15 s.

13. A process according to one of claims 1 to 12, characterized in that the pulse frequency is more than 0.1 Hz, preferably 1 Hz.

14. A process according to one of claims 1 to 13, characterized in that the pulse frequency is in the range 1 to 500 Hz.

15. A process according to one of claims 1 to 14, characterized in that the number of pulses received by each yeast, bacterium or mammalian cell is of the order of 1 to 100, preferably in the range 5 to 20.

16. A process according to one of claims 1 to 15, characterized in that the number of pulses received by each yeast, bacterium or mammalian cell is of the order of 10.

17. A process according to one of claims 1 to 16, characterized in that the incubation medium comprises at least one salt, preferably potassium phosphate.

18. A process according to one of claims 1 to 17, characterized in that the extraction medium comprises at least one agent that maintains the stability of the extracted proteins.

19. A process according to one of claims 1 to 18, characterized in that the extraction medium comprises at least one dithiothreitol reducing agent.

20. A process according to one of claims 1 to 19, characterized in that the electropulsing medium is deionized water for the yeasts and bacteria.

21. A process according to one of claims 1 to 20, characterized in that the profile of the electric field pulses is a square, trapezoidal, triangular, sinusoidal, single or half-wave rectified sinusoidal wave, or a wave with an exponential decay, unipolar, bipolar, symmetrical or asymmetrical.

22. A process according to one of claims 1 to 21, characterized in that the pulse profile is a square wave.

23. Proteins obtained by the process according to any one of claims 1 to 22.

24. Compositions comprising at least one protein obtained by the process according to any one of claims 1 to 22.
